(19) United States
(12) Patent Application Publication
(10) Pub. No.: US 2008/0045467 A1
(43) Pub. Date: Feb. 21, 2008

(54) Composition Containing Ginsenoside F1 Or Compound K For Skin External Application

(75) Inventors: Su-Nam Kim, Suwon-si (KR); Byung Young Kang, Seoul (KR); Su-Jong Kim, Yongin-si (KR); So-Hee Lee, Seoul (KR); Gyu-Ho Choi, Yongin-si (KR); Byeong-Gon Lee, Suwon-si (KR); Han-Kon Kim, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 10/586,973

(22) PCT Filed: Jun. 1, 2004
(86) PCT No.: PCT/KR04/01305

§ 371 Date: May 15, 2007

(30) Foreign Application Priority Data
Jan. 26, 2004 (KR) 10-2004-0004714

(51) Int. Cl. A61K 31/351; A61P 17/00; C07H 15/24

(52) U.S. Cl. 514/26; 536/6

(57) ABSTRACT

The present invention relates to an inhibitor for the biosynthesis of gelatinase comprising ginsenoside F1 (20-O-β-D-glucopyranosyl-20(S)-protopanaxatriol) or compound K (20-0-β-D-glucopyranosyl-20(S)-protopanaxadiol), which is a chief metabolite of ginseng saponin, as an active ingredient; and a cosmetic/medical composition for the prevention of skin-aging comprising the same which is superior in inhibiting the decomposition of epidermal-dermal junction and also in accelerating the generation thereof.

### TECHNICAL FIELD

The present invention relates to an inhibitor of the biosynthesis of gelatinase comprising ginsenoside F1 (20-O-β-D-glucopyranosyl-20(S)-protopanaxatriol) or compound K (20-O-β-D-glucopyranosyl-20(S)-protopanaxadiol), which is a chief metabolite of ginseng saponin, as an active ingredient; and to a cosmetic/medical composition for the prevention of skin-aging comprising the same that is superior in inhibiting the decomposition of the junction between the epidermis and dermis (hereinafter, “epidermal-dermal junction”) and also in accelerating the generation thereof.

### BACKGROUND ART

Skin is a primary protector of the human body and protects various organs of the body from the outside stimuli such as changes in temperature and humidity, UV light, and environmental pollution, and plays an important role in maintaining homeostasis such as regulation of body temperature. However, excessive physical/chemical stimuli from outside, UV light, stress and malnutrition decrease the normal function of skin, and accelerate skin aging effect such as loss of elasticity, keratinization, and formation of skin wrinkle. Especially, epidermal-dermal junction is severely damaged by UV light.

According to studies on the change in relation to the skin aging, a structural change of the epidermal-dermal junction exposed to UV light, such as multiplexing (the junction being decomposed to be multi-layer) or separation of the junction, is already present in the late twenties. Further, according to studies using the mouse model induced by long-term radiation of UVB, it was revealed that gelatinase formed on the epidermis by UVB radiation may give rise to damage of the epidermal-dermal junction, damage of the dermis and formation of skin wrinkle. However, such change is hardly found in skin that is not exposed much to UV light, even in the case of aged people. From such a result of the above studies, it can be assumed, that in the epidermis exposed to UV light, the formation of gelatinase (MMP-2, -9) is induced, which is involved in the damage of the epidermal-dermal junction.

Gelatinase (MMP-2, -9) is an enzyme that decomposes Type IV collagen, Type VII collagen and extracellular matrix, which are components of the epidermal-dermal junction. According to an assay using gelatin zymography and ELISA, both MMP-2 and MMP-9 were detected in the epidermis exposed to UV light. In addition, according to the above assay, it was revealed that gelatinase is present in the stratum basale and stratum spinosum of frozen skin of forehead, and is also present in the epidermis of the face exposed to UV light. Thus it can be assumed that UV light induces the formation of gelatinase in the stratum basale epidermis, a part of UV light is involved in the destruction of the dermis junction, and the rest of the UV light reaches the epidermis.

According to studies made using mice with long-term radiation of UV light, in the case of the long term radiation of UVB, gelatinase activity was detected on the whole layer of the epidermis. The damage to the epidermal-dermal junction was detected after the 5^{th} week from UVB radiation, and the degree of the damage was increased at the 7^{th} and the 10^{th} weeks. Especially at the 10^{th} week, separation of the epidermal-dermal junction and multiplexing of a part of the junction was detected. In addition, at the collagen fiber in the papillary layer of the dermis, the decrease in the fiber density was detected due to the long-term radiation of UVB. Therefore, it was assumed that the induced gelatinase is involved in the damage to the epidermal-dermal junction and the decomposition of collagen (see the 9^{th} FJ seminar, p12˜15, 2002).

When the epidermal-dermal junction is damaged, flattening, multiplexing or separation of the junction may occur and result in wrinkle formation, drooping skin and high probability of injury. In addition, when its inherent function as a barrier is lost, the epidermal-dermal junction cannot filter contaminants from the outside environment, thus the contaminants may be highly penetrating the dermis layer, leading to damage of the skin. In order to recover the damaged epidermal-dermal junction or keep in a healthy state, the constituents of the junction should firstly be kept. It is reported that the biosynthesis of Type IV collagen, Type VII collagen and laminin 10/11 decreases as age increases, and there is no change in laminin 5, and the biosynthesis of gelatinase (MMP-2, MMP-9) is increased (Lavker et al, J. Invest. Derm. 1979, 73:59-65; Pouliot et al. Exp. Dermatol. 2002, 11:387-397).

In order to prevent skin aging due to UW light or outer stress and to keep healthy and resilient skin, there have been efforts to keep the inherent function of the skin and activate skin cells to inhibit skin aging by using cosmetics enforced with bioactive ingredients from various animals, plants or microorganisms.

For the raw material of the cosmetics, it is desired to have anti-skin aging effects without any side effects. For this, there has been much interest in ginseng extracts, and steady studies have been done towards this. According to the studies on the ginseng extracts so far, study has developed from the extraction of ginseng saponin from ginseng extract to the preparation of ginseng aglycon and preparation/isolation/purification of ginsenoside F1 or compound K, chief metabolites in the human body, via purification of ginseng saponin.

Ginseng saponin has a triterpene structure of danunaraim type having sugars such as glucose, rhamnose, xylose and arabinose linked via ether bond with alcoholic OH moiety at the positions R1, R2 and R3 of the triterpene, and 29 types of saponin have been isolated from ginseng. Shibata, in 1964, named the components of ginseng saponin as “ginsenoside”, which refers to glycoside contained in ginseng. Ginsenosides are classified into ginsenoside-Ro, which is a family of oleanane saponin, and ginsenoside-Ra, -Rb1, -Rb2, -Rc, -Rd, -Re, -Rf, -Rg1, -Rg2, -Rg3 and -Rh, according to the order of movement in separation by TLC (thin-layer chromatography).

It was revealed that Ginseng saponin has a different structure from other saponins contained in plants of 750 kinds, and exhibits different pharmacological efficacies. Especially, ginseng saponin has been found to have very mild drug properties, no toxicity when used in excessive amounts, and no hemolysis. In addition, in order to use ginseng saponin as raw material for the anti-skin aging products, bioconverted ginseng aglycon, in which the efficacy of ginseng saponin is retained and the skin penetration ability is increased, was prepared and its efficacy was proved.

Examples of using the above ginseng extracts and ginseng saponin are disclosed in U.S. Pat. Nos. 5,565,207, 567,419, 5,578,312, 5,663,160, 5,626,868, 5,753,242, 5,747,300, 5,853,705, 6,027,728, 6,063,366, 6,221,372, and 6,228,378 (used in cosmetics), U.S. Pat. Nos. 5,569,459, 5,571,516, 5,587,167, 5,674,488, 5,665,393, 5,629,316, 5,776,460, 5,739,165, 5,916,555, 6,071,521, 6,083,512, and 6,255,313 (used in medicines), U.S. Pat. Nos. 5,591,611, 5,591,612, 5,736,380, 5,789,392, 5,780,620, 5,922,580, 5,935,636, 6,132,726, 6,156,817, and 6,207,164 (for the isolation/purification thereof).

However, ginseng saponin is very hydrophilic because it has a structure of dammaran type having sugars linked via ether bond with alcoholic OH moiety at positions R1, R2 and R3. Besides such a hydrophilic property of ginseng saponin, as its molecular weight increases, its skin penetration and absorption ability decreases. Thus ginseng saponin cannot go through the stratum cornieum of skin and there is a difficulty in introducing ginseng saponin into the skin. In a recent study on saponin metabolites, it was suggested that the pharmacological efficacy of ginseng saponin is due to the metabolites decomposed by human intestinal bacteria, not to the saponin itself. Further, it was found that, among the components of ginseng saponin, ginsenoside Rh1, Rh2, F1 and compound K, which have structures of aglycon having one sugar (glucose), have a pharmacological effects such as inhibiting the proliferation of cancer cells, inhibiting the proliferation of tumors, and increasing the efficacy of anti-cancer medicines.

Accordingly, studies on the formulation of ginsenoside F1 and compound K, which were obtained by removing a part of sugar from ginseng saponin, have been conducted in order to effectively introduce them to skin, and their anti-skin aging effects such as effect on the cell proliferation and the biosynthesis of collagen were proven (Korean Publications No. 2003-00601, No. 2003-0060018).

With regard to the invention for the epidermal-dermal junction, Korean Publication No. 2003-0066912 entitled “cosmetic composition for anti-aging” (Desmodium podocarpum DC. Extract), Korean Publication No. 2002-0019920 entitled “agents promoting the formation of skin basement membrane, agents promoting the formation of artificial skin and process for producing artificial skin”, Japanese Publication No. 2003-226655 entitled “composition containing laminin-5 production promoter and intergrin-α 6β 4 production promoter”, Japanese Publication Nos. 2003-183121, 2002-338460 entitled “composition for activating skin basal membrane”, Japanese Publication No. 2001-269398 entitled “skin basement membrane formation accelerator, artificial skin formation accelerator, and method of manufacturing for artificial skin” and Japanese Publication No. 2003-513220 entitled “Laminin-5 and the formation of basement membrane structure” have been disclosed. Therein, Desmodium podocarpum DC. Extract, komenuka oil and phenylpropanoid were used for the revival of the epidermal-dermal junction. However, for the ginseng components, there has been no report on their use for inhibiting the decomposition and accelerating the formation of epidermal-dermal junction, and for improving the skin wrinkle and skin elasticity.

### DISCLOSURE OF INVENTION

Under these circumstances, the present inventors studied on effective methods for controlling the various factors to the skin-aging, and found that a composition for external application containing ginseng ginsenoside F1 or compound K can inhibit or recover the denaturation, separation or multiplexing of the epidermal-dermal junction due to natural aging and photo aging. In other words, as ginsenoside F1 and compound K can inhibit the decomposition of the epidermal-dermal junction and accelerate the formation thereof, they can enforce the coherence between the epidermis and dermis. Accordingly, the present inventors developed a cosmetic/medical composition containing these substances that can be used for the prevention of skin aging, and thereby completed the present invention.

Therefore, the present invention provides a composition for external application for the prevention of skin aging comprising ginsenoside F1 and/or compound K. In addition, the present invention provides a method for preventing skin aging by using ginsenoside F1 and/or compound K so as to inhibit the decomposition of the epidermal-dermal junction and reinforce the cohesion between the epidermis and dermis.

More specifically, the present invention provides an inhibitor of the biosynthesis of gelatinase comprising ginsenoside F1 (20-O-β-D-glucopyranosil-20(S)-protopanxatriol) or compound K (20-O-β-D-glucopyranosil-20(S)-protopanxadiol), the chief metabolites of ginseng, as an effective ingredient; and a cosmetic/medical composition for the prevention of skin aging containing the same, which has an excellent effect in inhibiting the decomposition of the epidermal-dermal junction and accelerating the synthesis thereof.

The present invention provides a composition for external application containing ginsenoside F1, compound K or a mixture thereof.

The present invention provides an inhibitor of the biosynthesis of gelatinase containing at least one of ginsenoside F1 and compound K. The gelatinase is preferably MMP-2 or MMP-9.

When using the external application containing the gelatinase inhibitor according to the present invention, the epidermal-dermal junction can be protected. Protection of the epidermal-dermal junction may lead to the prevention of skin aging and improvement of skin wrinkle and skin elasticity.

### DETAILED DESCRIPTION OF INVENTION

Hereinafter, the present invention is described in more detail.

In the present invention, ginsenoside F1 or compound K can be contained in an amount of 0.001˜10% by weight, preferably 0.01˜5% by weight based on the total weight of the inhibitor or the composition.

The structures of ginsenoside F1 and compound K used in the present invention are represented by the following formulae 1 and 2, respectively.

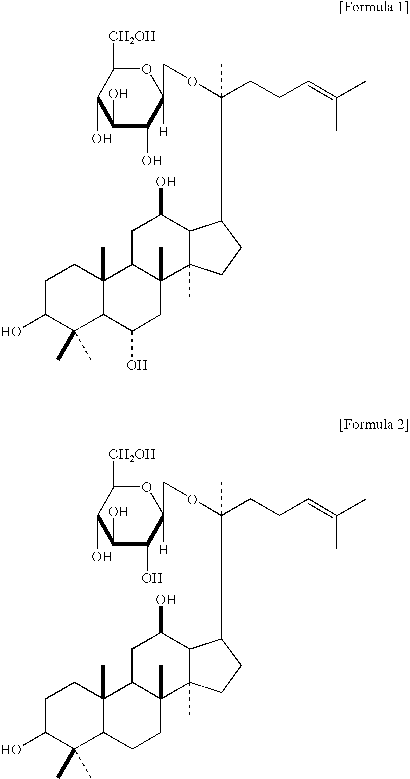

Ginsenoside F1 and compound K used in the present invention can be prepared by hydrolyzing purified ginseng saponin with acid, alkali or enzyme to remove sugar from ginseng saponin and then subjecting to the resultant silica gel column. The enzyme that can be used is an exo-sugar linkage decomposing enzyme such as β-glucosidase, α,β-arabinosidase or α,β-rhamnosidase which decomposes the sugar linkage of saponin, or a combined enzyme containing the same.

According to the present invention, various symptoms caused by skin aging, such as wrinkle or loss of skin elasticity, can be improved by using ginsenoside F1 and compound K to protect the epidermal-dermal junction. The usage amount of ginsenoside F1 and compound K in total is 0.001˜10% by weight, preferably 0.0˜5% by weight based on the total weight of the composition, according to the in vitro and in vivo experiments.

The composition for external application according to the present invention is to improve skin wrinkles and elasticity, and it may be formulated into, but is not limited thereto, skin softener, nutrition water, massage cream, nutrition cream, pack, gel, lotion, ointment, cream, patch or spray.

In addition, in each formulation of the composition, components other than the above essential components can be easily selected and added by a person skilled in the art depending on the formulation or use object of the composition.

### BEST MODE FOR CARRYING OUT THE INVENTION

The present invention will be described in more detail by way of the following examples and experimental examples. However, these examples are provided for the purpose of illustration only and should not be construed as limiting the scope of the invention, which will be apparent to one skilled in the art.

### REFERENCE EXAMPLE 1

### Purification of Ginseng Saponin

2 kg of red ginseng (KT&G Corporation; 6 years old red ginseng) was added to 4 L of ethanol containing water, refluxed 3 times at 77° C. then deposited at 15° C. for 6 days. Residues and remainders were separated by filtration and centrifugation, and the remainders were concentrated under reduced pressure to obtain extract. The extract was suspended into water and extracted 5 times with 1 L of ether to remove pigments, and the aqueous layer thereof was extracted 3 times with 500 ml of 1-butanol. The obtained 1-butanol layer was treated with 5% KOH, washed with distilled water and concentrated under reduced pressure to obtain 1-butanol extract. 1-butanol extract was dissolved in a small amount of methanol, and a large amount of ethyl acetate was added thereto to obtain precipitate. The precipitate was dried to obtain 100 g of purified ginseng saponin (yield: 5%). The same operation was repeated 10 times to obtain 1 kg of purified saponin.

The extraction/identification of ginsenoside F1 and compound K in the following examples was made in accordance with Korean Patent application No. 20001-67964 (Publication No. 10-2003-0037005).

### EXAMPLE 1

### Preparation of Ginsenoside F1 by Acid Hydrolysis

100 g of purified ginseng saponin obtained in Reference Example 1 was dissolved in 20 times amount (v/w) of sulfuric acid/50% ethanol solution (v/w), and heat-refluxed in a water bath at 100° C. for 6 hours to hydrolyze sugar linkage of saponin. The reactant was concentrated under reduced pressure to remove any solvent, and the residue was suspended into 1,000 ml of distilled water. It was then extracted with ether 3 times, each time using an equal amount. The whole ether layer was washed with distilled water, and then dried with anhydrous MgSO₄, filtered and concentrated to obtain crude product. The obtained crude product was separated with silica gel column chromatography (separated by increasing the polarity in the manner of changing the ratio of chloroform: methanol from 9:1 to 4:1). Each aliquot underwent thin film chromatography (chloroform/methanol/water=65/35/10, R_{f}=0.65) to separate aliquot of ginsenoside F1, and 2400 mg of ginsenoside F1 was finally obtained (yield: 2.4%).

### EXAMPLE 2

### Preparation of Compound K by Enzymatic Hydrolysis

100 g of purified ginseng saponin obtained in Reference Example 1 was dissolved in citrate buffer solution (pH 5.5). Thereto was added 1 g of naringinase, separated from Penicillium sp., then the mixture was reacted in a water bath at 40° C. with stirring for 48 hours. Checking periodically with thin film chromatography, when the substrate was completely removed, the reaction was terminated by heating for 10 minutes in a hot water bath. The reactant was then extracted 3 times with ether, each time using an equal amount of ether, and concentrated. The obtained product was separated with silica gel chromatography (separated by increasing the polarity in the manner of changing the ratio of chloroform: methanol from 9:1 to 4:1). Each aliquot underwent with thin film chromatography (chloroform/methanol/water=65/35/10, R_{f}=0.73) to separate the aliquot of compound K, and 4400 mg of compound K was finally obtained (yield 4.4%).

### EXPERIMENTAL EXAMPLE 1

### Efficacy in Inhibiting the Biosynthesis of Gelatinase A (MMP-9) and Gelatinase B (MMP-2) When Irradiated with UV Light

Human keratinocyte was cultured with a concentration of 10⁴ cells/well in 24-well plate mediums, and 24 hours later they were irradiated with 30 mJ/cm² of UVB. Each medium was then exchanged with a new one containing the compound separated in either Example 1 or 2 at the densities of 0.1 ppm, 1 ppm or 10 ppm, respectively. After 2 days of cultivation, the supernatants were obtained and gelatin zymography was performed on the obtained supernatant to form MMP-2 and MMP-9. The amount of MMP-2 and MMP-9 thus formed was determined by densitometer for each medium, and was compared with the amount of MMP-2 and MMP-9 in the control group, which was set as 100 (The mediums not containing the compounds in Examples 1 and 2 were cultured as a control group). The results are shown in Table 1.

TABLE 1

| Component | Density (ppm) | MMP-2 (%) | MMP-9 (%) |
|---|---|---|---|
| Example 1 | 10 | 51 | 55 |
| | 1 | 67 | 69 |
| | 0.1 | 82 | 85 |
| | Control group | 100 | 100 |
| Example 2 | 10 | 48 | 49 |
| | 1 | 61 | 62 |
| | 0.1 | 79 | 78 |
| | Control group | 100 | 100 |

From the results in Table 1, it can be seen that the composition of the present invention containing ginsenoside F1 and/or compound K can inhibit the biosynthesis of MMP-2 and MMP-9, which are the enzymes decomposing skin component Type IV collagen and Type VII collagen, and thereby can prevent decomposition of the epidermal-dermal junction.

### EXPERIMENTAL EXAMPLE 2

### Efficacy in Biosynthesis of Type IV Collagen of Skin Cell

Human keratinocyte was cultured with a concentration of 5×10⁴ cells/well in 24- well plate mediums, and each medium was exchanged with a new one containing the compound separated in either example 1 or 2 at the densities of 0.1 ppm, 1 ppm or 10 ppm. After 24 hours of cultivation, the supernatants were harvested and the amount of Type IV collagen was quantified using Dot Blot method. The amount was compared with the amount at the control group, which was set as 100, and the results are shown in Table 2.

TABLE 2

Biosynthesis of Type IV collagen (%)

| Density (ppm) | Example 1 | Example 2 |
|---|---|---|
| 10 | 134 | 139 |
| 1 | 129 | 128 |
| 0.1 | 116 | 119 |
| Control | 100 | 100 |

From the results in Table 2, it can be seen that the composition of the present invention containing ginsenoside F1 and/or compound K can increase the biosynthesis of Type IV collagen in a density-dependent manner.

### EXPERIMENTAL EXAMPLE 3

### Efficacy in Biosynthesis of Type VII Collagen of Skin Cell

Human fibroblast was cultured with a concentration of 10⁴ cells/well in 24-well plate mediums, and each medium was exchanged with a new one containing the compound separated in Example 1 or 2 at the densities of 0.1 ppm, 1 ppm or 10 ppm. After 24 hours of cultivation, the supernatants were harvested and the amount of Type VII collagen was quantified using Dot Blot method. The amount was compared with that of the control group, which was set as 100, and the results were shown in Table 3.

TABLE 3

Biosynthesis of Type VII collagen (%)

| Density (ppm) | Example 1 | Example 2 |
|---|---|---|
| 10 | 141 | 146 |
| 1 | 132 | 135 |
| 0.1 | 118 | 120 |
| Control group | 100 | 100 |

### EXPERIMENTAL EXAMPLE 4

### Efficacy in Biosynthesis of Laminin 10/11 of Skin Cell

Human keratinocyte was cultured with a concentration of 5×10⁴ cells/well in 24-well plate mediums, and each medium was exchanged with a new one containing the compound separated in either Example 1 or 2 at the densities of 0.1 ppm, 1 ppm or 10 ppm. After 24 hours of cultivation, the supernatants were harvested and the amount of laminin 10/11 was quantified using Dot Blot method. The amount was compared with that of the control group, which was set as 100, and the results are shown in Table 4.

TABLE 4

Biosynthesis of laminin 10/11 (%)

| Density (ppm) | Example 1 | Example 2 |
|---|---|---|
| 10 | 134 | 137 |
| 1 | 125 | 125 |
| 0.1 | 112 | 115 |
| Control group | 100 | 100 |

### EXPERIMENTAL EXAMPLE 5

### Determination of Change in Epidermal-Dermal Junction of Nude Mouse

In order to verify the change to the epidermal-dermal junction irradiated with UV light by the composition of the present invention, external applications with the formulation of nutrition cream were prepared according to the ratios in Table 5.

TABLE 5

| | Formulation (wt %) | | | |
|---|---|---|---|---|
| Component | 1 | 2 | 3 | Comparative |
| Distilled water | To 100 | To 100 | To 100 | To 100 |
| Ginsenoside F1 | 0.1 | — | 0.1 | — |
| Compound K | — | 0.1 | 0.1 | — |
| Vegetable oil | 1.50 | 1.50 | 1.50 | 1.50 |
| Stearic acid | 0.60 | 0.60 | 0.60 | 0.60 |
| Glycerol stearate | 1.00 | 1.00 | 1.00 | 1.00 |
| Stearyl alcohol | 2.00 | 2.00 | 2.00 | 2.00 |
| Polyglyceryl-10 pentasterate | 1.00 | 1.00 | 1.00 | 1.00 |
| & Behenyl alcohol | | | | |
| Arachidyl behenyl alcohol & | 1.00 | 1.00 | 1.00 | 1.00 |
| arachidyl glucoside | | | | |
| Cetearyl alcohol & cetearyl | 2.00 | 2.00 | 2.00 | 2.00 |
| glucoside | | | | |
| PEG-100 stearate & | 1.50 | 1.50 | 1.50 | 1.50 |
| glycerol oleate & | | | | |
| propyleneglycol | | | | |
| Caprylic/capric triglyceride | 11.00 | 11.00 | 11.00 | 11.00 |
| Cyclomethicon | 6.00 | 6.00 | 6.00 | 6.00 |
| Preservative, perfume | q. s. | q. s. | q. s. | q. s. |
| Triethanolamine | 0.1 | 0.1 | 0.1 | 0.1 |

Each of the formulations 1˜3 and the comparative formulation 1 were applied to the back of each nude mouse 5 times in a week for 2 weeks, and for 12 weeks thereafter each of the formulations 1˜3 and the comparative formulation 1 were applied 5 times per week while irradiating UV light 3 times. After biopsy, the change of the epidermal-dermal junction was determined by electron microscope.

As a result, it was found that in the case of applying ginsenoside F1 and compound K while irradiating with UV light, there was little change, separation or multiplexing of the epidermal-dermal junction, compared to the case of applying the comparative formulation 1. Therefore, it was verified that ginsenoside F1 and compound K can diminish skin wrinkles and reinforce skin elasticity. The results are shown in Table 6.

TABLE 6
Change of epidermal-dermal junction

| | Formulation | Formulation | Formulation | Comparative | |
|---|---|---|---|---|---|
| | 1 | 2 | 3 | formulation 1 | Untreated |
| Multiplexing | ++ | ++ | + | ++++ | +/− |
| Cutting | ++ | ++ | + | ++++ | +/− |

(+/−: Little, +: low, ++: middle-lower, +++: middle-upper, ++++: high)

### EXPERIMENTAL EXAMPLE 6

### Improvement of Skin Wrinkle in Human (Image Analysis)

The following was performed to identify the effect of the above formulations in Table 5 on the improvement of skin wrinkle. Eighty women aged in 30-39 were divided into 4 groups (20 people per each group: i.e. one group for each formulation 1, 2 and 3 and comparative formulation 1). To each group, the formulation 1, 2, 3 or comparative formulation 1 was applied once every day for 8 weeks, and replicas were prepared after 8 weeks using silicon. The state of skin wrinkles was image analyzed by visiometer (SV600, Courage+Khazaka Electronic GmbH, Germany). The average of the values obtained by subtracting each parameter value before the application from the same person's corresponding parameter value 8 weeks later are shown in Table 7.

TABLE 7

| Clinical result 8 weeks after using | R1 | R2 | R3 | R4 | R5 |
|---|---|---|---|---|---|
| Comparative formulation 1 | 0.28 | 0.27 | 0.22 | 0.04 | 0.04 |
| Formulation 1 | −0.21 | −0.21 | −0.12 | −0.04 | −0.03 |
| Formulation 2 | −0.20 | −0.22 | −0.12 | −0.03 | −0.02 |
| Formulation 3 | −0.20 | −0.20 | −0.12 | −0.04 | −0.04 |

R1: Difference between the maximum value and the minimum value of skin wrinkle contour
R2: Average of R1 obtained by the difference between the value of an arbitrary contour and the value of the fifth contour from the arbitrary contour
R3: Maximum of R1 obtained by the difference between the value of an arbitrary contour and the value of the fifth contour from the arbitrary contour
R4: Average of each peak-to-peak value at the baseline of wrinkle contour
R5: Difference between the baseline and the value of each wrinkle contour

As can be seen in the above Table 7, the formulations 1˜3 are superior in improvement of skin wrinkle, and especially the effect of formulation 3 was excellent.

### EXPERIMENTAL EXAMPLE 7

### Improvement of Skin Elasticity in Human Body

The effect of the nutrient cream prepared according to the above Table 5 on the improvement of skin elasticity was determined. Forty healthy women in more than 30 years old were divided into 4 groups, and for each group the formulations 1, 2 or 3, or comparative formulation 1 was applied to the faces in each group twice every day for 12 weeks at a temperature of 24˜26° C. and 75% RH. The skin elasticity was determined using Cutometer SEM 575 (C+K Electronic Co., Germany), and the result is shown in Table 8. In Table 8, the result is described by Δ R8 (=R8(left)-R8 (right)) in Cutometer SEM 575. In this regard, R8 means viscoelasticity of skin. Besides, subjective assessment of the effect was also made by a questionnaire after the experiment,

TABLE 8
Skin elasticity

| Product | viscoelasticity |
|---|---|
| Comparative Formulation 1 | 0.11 |
| Formulation 1 | 0.42 |
| Formulation 2 | 0.42 |
| Formulation 3 | 0.44 |

As can be seen in Table 8, the skin elasticity improved in the group applied with formulations 1, 2 and 3 comprising ginsenoside F1 and/or compound K, especially for formulation 1, more than in the group applied with comparative formulation 1.

In addition, the superior skin elasticity with formulations 1˜3, especially formulation 3, was also proved by the questionnaire. The results are shown in Table 9 below.

TABLE 9
Result of the questionnaire on the improvement of skin elasticity

| | Number of Respondent | | | |
|---|---|---|---|---|
| Group | Very good | good | middle | Bad |
| Comparative formulation | 1 | 3 | 3 | 3 |
| Formulation 1 | 3 | 4 | 2 | 1 |
| Formulation 2 | 3 | 5 | 2 | 0 |
| Formulation 3 | 4 | 5 | 1 | 0 |

Other examples of formulation according to the present invention are described in the following; however, the formulation of the external application containing ginsenoside F1 and/or compound K according to the present invention is not limited thereto. All of them have efficacy in inhibiting the biosynthesis of gelatinase.

### [Formulation 4] Skin Softener (Skin Lotion)

| Component | wt % |
|---|---|
| Distilled water | To 100 |
| Ginsenoside F1 | 0.1 |
| Compound K | 0.1 |
| Butylene glycol | 2.0 |
| Propylene glycol | 2.0 |
| Carboxyvinyl polymer | 0.1 |
| PEG-12 nonyl phenyl ether | 0.2 |
| Polysorbae 80 | 0.4 |
| Ethanol | 10.0 |
| Triethanolamine | 0.1 |
| Preservative, pigment, perfume | q.s. |

### [Formulation 5] Nutrition Water (Milk Lotion)

| Component | wt % |
|---|---|
| Distilled water | To 100 |
| Ginsenoside F1 | 0.1 |
| Compound K | 0.1 |
| Beeswax | 4.0 |
| Polysorbae 60 | 1.5 |
| Sorbitan sesquioleate | 1.5 |
| Liquid paraffin | 0.5 |
| Caprylic/capric triglyceride | 5.0 |
| Glycerine | 3.0 |
| Butylene glycol | 3.0 |
| Propylene glycol | 3.0 |
| Carboxy vinyl polymer | 0.1 |
| Triethanolamine | 0.2 |
| Preservative, pigment, perfume | q. s. |

### [Formulation 6] Nutrition Cream

| Component | wt % |
|---|---|
| Distilled water | To 100 |
| Ginsenoside F1 | 0.1 |
| Compound K | 0.1 |
| Beeswax | 10.0 |
| Polysorbate 60 | 1.5 |
| PEC 60 Hydrogenated caster oil | 2.0 |
| Sorbitan sesquioleate | 0.5 |
| Liquid paraffin | 10.0 |
| Squalane | 5.0 |
| Caprylic/capric triglyceride | 5.0 |
| Glycerine | 5.0 |
| Butylene glycol | 3.0 |
| Propylene glycol | 3.0 |
| Triethanolamine | 0.2 |
| Preservative, pigment, perfume | q. s. |

### [Formulation 7] Massage Cream

| Component | wt % |
|---|---|
| Distilled water | To 100 |
| Ginsenoside F1 | 0.1 |
| Compound K | 0.1 |
| Beeswax | 10.0 |
| Polysorbate 60 | 1.5 |
| PET 60 Hydrogenated caster oil | 2.0 |
| Sorbitan sesquioleate | 0.8 |
| Liquid paraffin | 40.0 |
| Squalane | 5.0 |
| Capiylic/capric triglyceride | 4.0 |
| Glycerine | 5.0 |
| Butylene glycol | 3.0 |
| Propylene glycol | 3.0 |
| Triethanolamine | 0.2 |
| Preservative, pigment, perfume | q. s. |

### [Formulation 8] Pack

| Component | wt % |
|---|---|
| Distilled water | To 100 |
| Ginsenoside Fl | 0.1 |
| Compound K | 0.1 |
| Polyvinyl alcohol | 13.0 |
| Sodium carboxy methyl cellulose | 0.2 |
| Glycerine | 5.0 |
| Allantoin | 0.1 |
| Ethanol | 6.0 |
| PEG-12 nonyl phenyl ether | 0.3 |
| Polysorbate 60 | 0.3 |
| Preservative, pigment, perfume | q. s. |

As described in the above, the composition according to the present invention comprising at least one of ginsenoside F1 and compound K can inhibit the biosynthesis of gelatinase (MMP-2, MMP-9), increase the biosynthesis of Type IV collagen, Type VII collagen and laminin 10/11, and inhibit the decomposition of the epidermal-dermal junction and accelerate the formation thereof, thus improving the skin wrinkle and skin elasticity. Therefore, it can be used in the composition for external application having anti-skin aging efficacy.

1. An inhibitor of the biosynthesis of gelatinase comprising one or more of ginsenoside F1 and compound K as an active ingredient.

2. The inhibitor according to claim 1, wherein said gelatinase is MMP-2 or MMP-9.

3. A composition for external application comprising the inhibitor of the biosynthesis of gelatinase of claim 1 or 2.

4. The composition according to claim 3, wherein said composition is for inhibiting the decomposition of epidermal-dermal junction or for accelerating the synthesis thereof.

5. The composition according to claim 3, wherein said composition is for improving skin wrinkles or skin elasticity.
